(19) United States
(12) Patent Application Publication
(10) Pub. No.: US 2017/0056165 A1
(43) Pub. Date: Mar. 2, 2017

(54) Stent With Mechanical Or Biological Heart Valve For Minimally Invasive Valve Replacement Procedure And Stent Application Device

(71) Applicant: Antonio Francisco Neves Filho, Pindamonhangaba (BR)

(72) Inventor: Antonio Francisco Neves Filho, Pindamonhangaba (BR)

(21) Appl. No.: 15/118,841

(22) PCT Filed: Feb. 27, 2015
(86) PCT No.: PCT/BR2015/050024

§ 371 Date: Aug. 12, 2016

(30) Foreign Application Priority Data
Mar. 14, 2014 (BR) BR102014006114-2

(51) Int. Cl. A61F 2/24

(52) U.S. Cl. CPC A61F 2/2418; A61F 2/2409; A61F 2/2436; A61F 2230/008

(57) ABSTRACT

A cardiac stent assembly featuring support flaps for a heart valve replacement procedure, while being able to place a mechanical or biological valve in an adapter fixed on the inner part of a stent body of the stent assembly. The stent assembly can be placed within heart valves sustained on an annular or ovoid mechanical support ring. For the positioning procedure of the stent assembly in the valve, an application device can be used wherein the stent body is inside the application device so that is possible to assemble and keep the entire stent assembly ready for use.

This invention patent application discloses a cardiac stent with a mechanism for positioning the mechanical or biological valve to be used during heart valve replacement or procedure, as well as a stent application device thereof. The stent has a tubular shape made of grated material with two flaps, with the heart valve fixed within. The application device comprises a syringe inside which the stent is placed, said syringe comprising a threaded cap that allows storage of the stent in a solution for conservation. Two screws pierce through the plunger of the syringe and fix the stent structure.

### DESCRIPTION OF THE STATE OF THE ART

The state of the art features a variety of devices for heart valve replacement, especially, by stents. Some of these stents feature an application device, specifically designed for the positioning procedure.

The surgical approach of mitral valve pathologies demands the application of annuloplasty rings for fixing the base, which is a relatively time-consuming procedure, since anchoring of fixing wires is needed.

Document W02009134701 (A2) features a positioning, opening and fixing system of a device for mitral valve replacement, which comprises a grated material that, when released from the application device, opens one of the ends which adapts to the shape of the mitral valve. The end that remains in the ventricle is fixed by tension wires anchored to the myocardium. Similar constructions are featured in the US 2013/0172978 and WO 2013096541.

In this application, as well as the other mentioned documents, a specific application device was developed for this stent.

Differently, this invention comprises a cardiac stent for heart valve without using tension wires for fixing, in other words, this stent is fixed by flaps mounted in its own structure.

The state of the art features a variety of distinct approaches to the treatment of dysfunctions associated to heart valves. One traditional method comprises the use of mechanical side-flow valves, such as the Starr-Edwards (initially disclosed in patent U.S. Pat. No. 3,099,016). This device consists in a closure element—usually spherical—lodged in the path of the blood flow, diverting it. The initial project envisaged numerous disadvantages, such as structural fragility of the cage stems, low resistance of the sphere (causing injury to the septa) and the need of a permanent anticoagulant medicinal therapy for the patient. The position of the valves should also be highlighted as another impacting factor on patient health.

The evolution of occlusion devices took place through development of forward flow mechanical valves, comprising pivotal structures that enabled a unidirectional blood flow. However, negative aspects remained, such as mechanical fatigue, use of anticoagulant medication and calcification of the heart valve. Partial solutions for the problem of the state of the art were described in documents PI9700076-0 and U.S. Pat. No. 6,113,631.

An alternative proposal comprises the use of bioprosthesis or bioprosthetic valves, mainly built from biological tissue (bovine or pig pericardium), mimicking the leaflets that constitute human heart valves. Documents PI9202905-1 and U.S. Pat. No. 6,358,277 illustrate this concept.

On the other hand, the device revealed in document PI0711664-0 was developed to replace damaged tendinous cords in case of a mitral valve prolapse. There is no mention or any information in the report that supports its application in valve replacement. In this document, a rough connection using wires and a hook is featured. Similarly, the international application W02012106602 features an apparatus and a positioning method, incorporating some common elements to PI0711664-0.

The application US2013079873, in turn, describes a set comprising a prosthetic valve with a valve position and support, comprising fixing means for support to the mitral annulus, providing a closing mechanism in order to avoid slipping of the chains and mentioning the possibility of the fixing device being a screw. One of the limitations inherent to this device is the impossibility of timely adjustment of chain dimensions. In case adjustments are needed, the device must be taken off, adjusted and repositioned over the valve. Further, the exposure of fixing support elements facilitates contamination or even injury of cardiac muscles.

In view of the state of the art, this invention enables a new stent and application device concept, for heart valve replacement or exchange procedures, with higher reliability and lesser duration of the procedure and equipment costs.

### DESCRIPTION OF THE DRAWINGS

FIG. 1 shows that the application device may be introduced in the cardiac muscle in two ways. In both ways, the stent (3) is introduced by its application device (1 or 2), which may be positioned from the upper part of the heart, right atrium, left atrium or through the pulmonary artery (1), or, alternatively, from a cut on the lower part of the myocardium (2).

FIGS. 2A and 2B feature assemblies of the stents within the application devices, respectively, according to the introduction procedure of the application device (1) from the upper part (1) or the application device (2) from the lower part (2) of the myocardium. Both assemblies are needed so that the stent (3) is positioned correctly within the heart valve.

FIG. 2C highlights a screw (7) that pierces through the plunger (5) of the application device (1) to connect to the stent structure (3), preferably, on the upper flap (8) of the stent.

FIG. 3 shows an exploded view of the assembly of the stent (3) in which an adapter (12) is fixed, where a mechanical valve (11) is screwed or, alternatively, a biological valve (10).

FIGS. 4A, 4B and 4C show, respectively, the open stent (3), without a heart valve, with a biological valve (10) or a mechanical valve (11). FIG. 4D shows a cross-sectional view of the stent (3) with a mechanical valve (11).

### DETAILED DESCRIPTION OF THE DRAWINGS

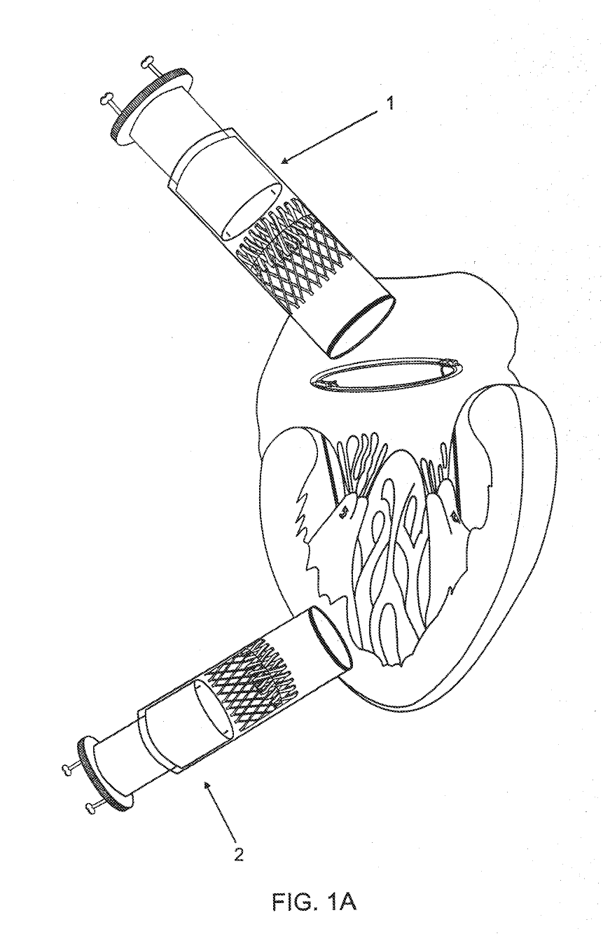

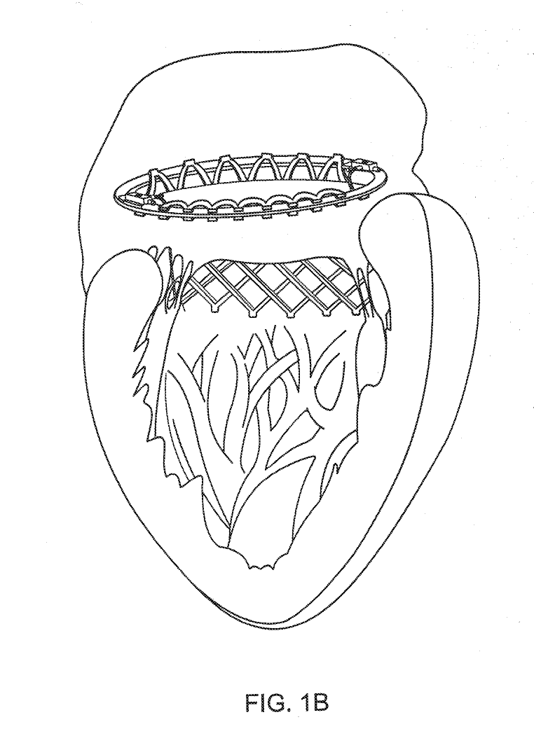

FIG. 1 shows, in its upper part, the application device (1) with the stent (3) mounted within in a stretched manner. When the application device is positioned and the stent (3) is released in the myocardium valve of the patient, the stent expands so that the upper flap (8) of the stent (3) remains above the myocardium valve, while the lower flap (9) remains below the myocardium valve.

On the same FIG. 1, a second procedure option is shown with the application device (2) from the lower part of the myocardium, so that the stent (3) is introduced by the application device.

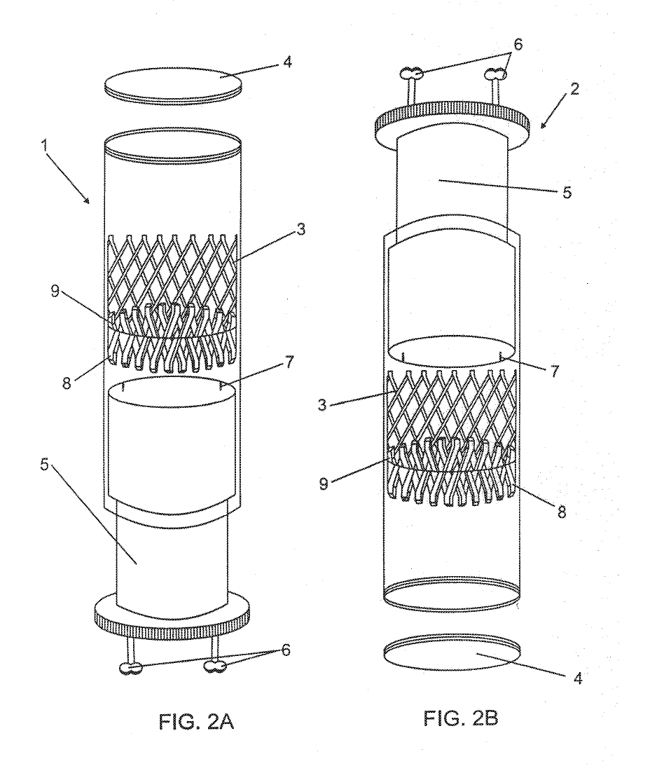

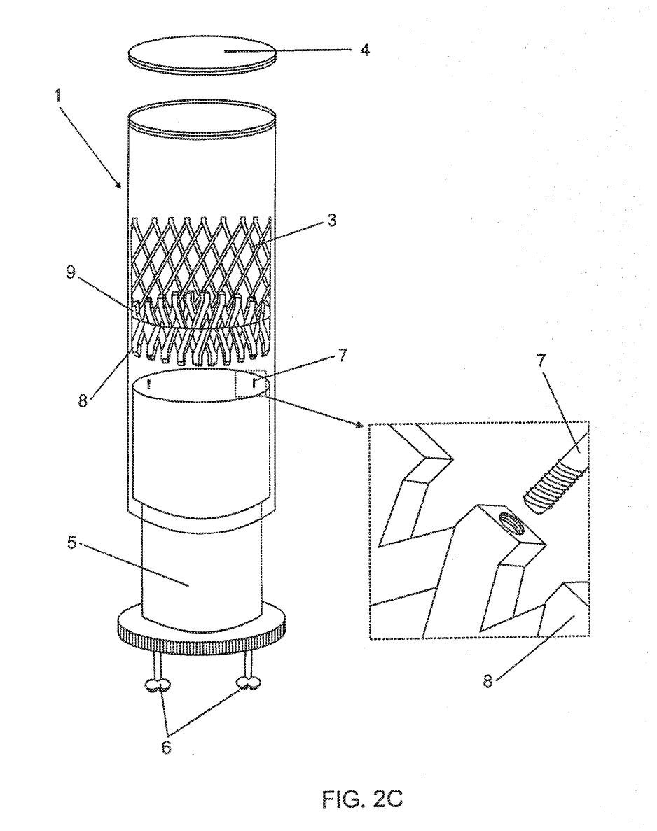

FIGS. 2A and 2B show the assembly of the stent (3) within application devices (1) and (2), so that the stent (3) is stretched and compressed within the application device. In assembly 2A, the stent structure (3) is through the upper flap (8) which is attached to the plunger (5) by a screw (7). In this embodiment, the application device must be introduced from the upper part of the myocardium. FIG. 2B shows a configuration in which the stent (3) is positioned within the application device (2) in reverse form to the assembly of FIG. 2A, which allows the same positioning of the stent (3) in the myocardium valve.

In summary, the position in which the stent (3) is assembled within the application device (1 or 2) is determined by the introduction point of the application device in the myocardium.

Additionally, the application devices (1) or (2) may be used as storage means of the stent (3). Accordingly, there is the option of a cap (4), which seals the application device, allowing it to be filled with a preservative liquid.

FIG. 2C highlights the fixing feature of the stent (3) on the screws (7) of the application device (1 or 2).

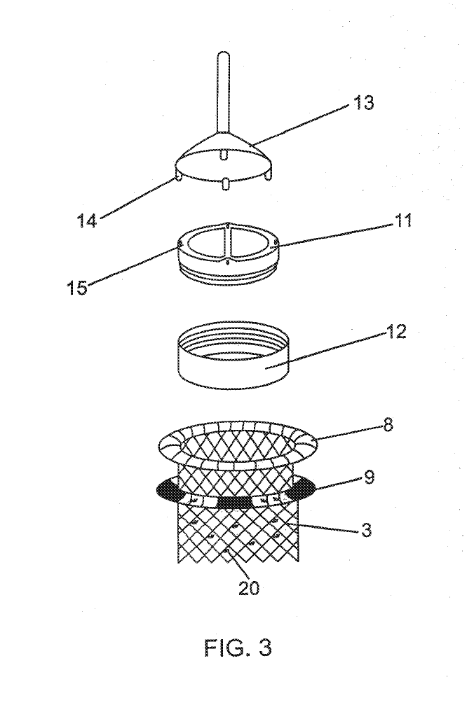

FIG. 3 features an exploded view of the stent (3) in an open position, an adapter (12) with a thread and a mechanical valve (11), which is fixed and guided by the movement device (13) which is attached to the valve (11) through fixing elements (14). Optionally, the mechanical valve (11) may be replaced by a biological valve (10).

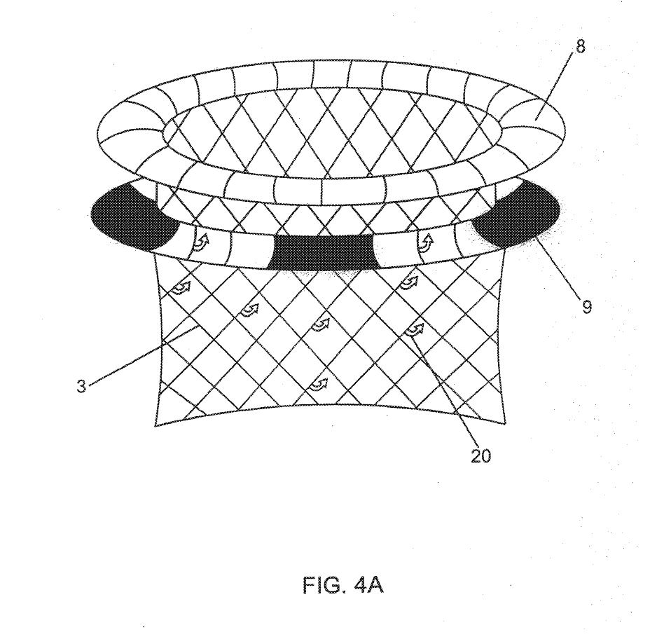

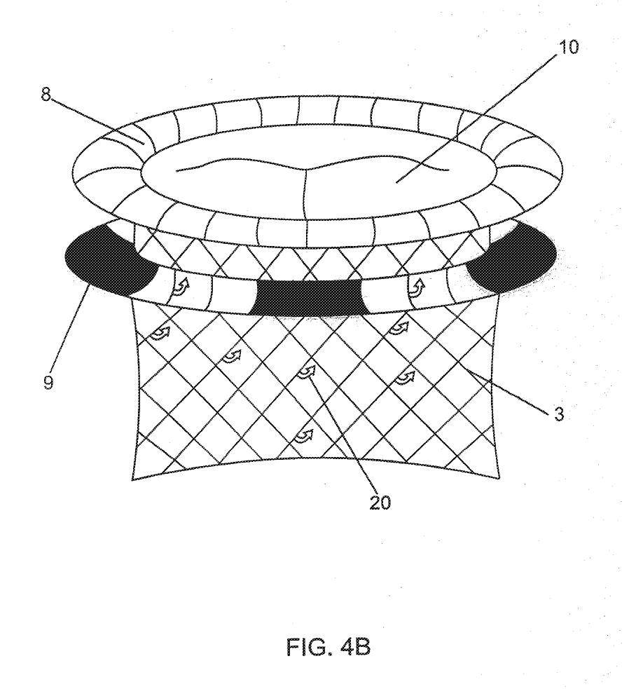

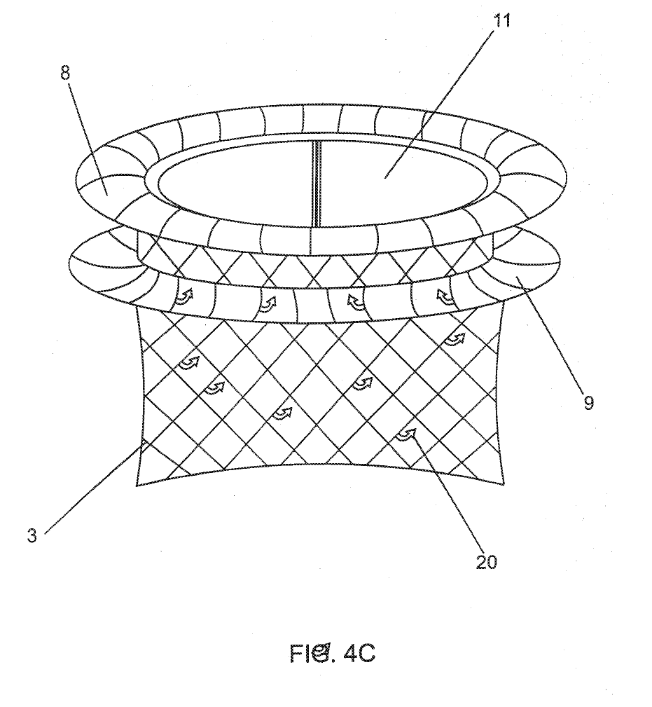

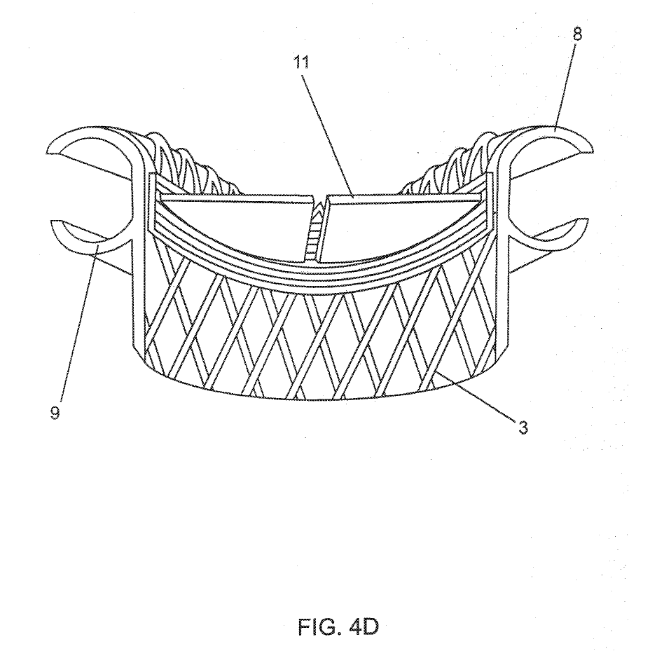

FIGS. 4A, 4B and 4C show the stents (3) in an open position, respectively, without valves, with the biological valve (10) and with the mechanical valve (11).

FIG. 4D shows the device, in a cross-sectional view, with the mechanical valve (11) installed in the stent body (3) with the upper flap (8) and (9) in the position of application to the heart valve.

### DESCRIPTION OF THE INVENTION

The device of this invention comprises a stent assembly for heart valve and application device thereof. The stent comprises a tubular structure with one of its ends including an upper flap (8) and a lower flap (9). When the stent (3) is released from the application device (1 or 2), the tubular structure expands, and the flaps are opened, fixing the stent (3) to the heart valve.

Due to its shape, the stent requires a specific application device (1 or 2) to be used. The application device (1 or 2) features a plunger (5) with two screws (6) that pierce through it. The adjustments of the screws (6), in one of the ends, are located on the outer part of the application device (1 or 2) and the threaded end (7) is on the inner part of the application device. In the threaded part (7), the stent (3) is attached and kept inside the application device, as seen in FIG. 2C. Preferably, the application device must have an anatomical shape, which facilitates intra-arterial movement until the inside of the cardiac muscle.

There is the option of keeping the application device assembled and sealed by a cap (4), allowing the stent (3) to be kept in a preservation solution, such as formaldehyde, for example.

Whichever the procedure, a plasty ring may be featured or a previously inserted heart valve, which facilitates, in these situations, mechanical support of the stent. Depending on how the procedure is carried out, the stent must be placed within the application device (1 or 2). This is because the flaps (8 and 9) of the stent (3) have to be positioned in the heart valve, while the stent body must remain in the ventricular part of the valve.

In case of an applying procedure from the upper part of the myocardium, the stent (3) must be assembled with the flaps near the plunger (5). In the procedure, from the lower part of the myocardium, the stent (3) must be assembled with the flaps (8 and 9) closer to the exit of the application device (2). With these two assemblies, the correct position of the stent (3) is ensured inside the heart valve. Furthermore, the stent (3) must have a valve in the inner part of its tubular structure. This valve must enable the blood flow to occur only on one direction: always from the atrium to the ventricle, avoiding backflow in the region.

The valve is assembled in an adapter (12) which, in turn, is fixed to the stent body (3), preferably, between the flaps (8 and 9).

The valve may be made of biological material (10) or mechanical (11). In case of a biological valve, the stent (3) may be compressed within the application device (1 or 2) and kept inside with a preservative solution. However, when mechanical valves (11) are used, the stent (3) must be stored without the mechanical valve (11), with only the upper flap (8) and lower flap (9) being compressed within the application device (1 or 2).

Alternatively, the stent may be stored without the mechanical valve (11), with the mechanical valve-adapter (9) assembly fixed inside the stent (3) during the surgical procedure.

1. A stent assembly comprising:
a stent body of tubular shape and grated structure;
an upper flap; and
a lower flap; and
an adapter;
wherein the lower flap is in proximity to and parallel to the upper flap, located at an intermediate part of the stent body;
wherein both the upper and lower flaps and the stent body are made of wired material and at least partially coated with an inert material, which allows physiological movement of the mitral annulus and fitting of the adapter; and
wherein the adapter is used to support a heart valve within the stent between both the upper and lower flaps.

2. The stent assembly according to claim 1 further comprising the heart valve being a mechanical valve with two floodgates that allow a unidirectional flow.

3. The stent assembly according to claim 1 further comprising the heart valve being a biological valve.

4. The stent assembly according to claim 1, wherein the stent has a radiographic opaque property, allowing it to be viewed through radiography.

5. The stent assembly according to claim 1 further comprising an oval or annuloplasty ring, with the ring placed between the upper flap and lower flap on an outer part of the stent body.

6. The stent assembly according to claim 1 further comprising:
a fixing system active through the upper flap and lower flap; and
claws at a lower part of the stent body.

7. (canceled)

8. The stent assembly according to claim 1, wherein at least portions of the stent body, the upper flap, and the lower flap have hooks.

9. The stent assembly according to claim 1, wherein at least portions of each of the upper flap and lower flap are made of metallic segments.

10. The stent assembly according to claim 1 further comprising an application device, wherein the application device comprises:
a cylindrical body;
a plunger; and
fixing elements;
wherein the plunger is located in the cylindrical body, and the plunger is attached to the stent body via the fixing elements;
wherein the stent body is stretched and elongated inside the cylindrical body; and
wherein the end of the stent body in proximity to the upper flap and lower flap are facing the side of the plunger.

11. The stent assembly according to claim 1 further comprising an application device, wherein the application device comprises:
a cylindrical body;
a plunger; and
fixing elements;
wherein the plunger is located in the cylindrical body, and the plunger is attached to the stent body via the fixing elements;
wherein the stent body is stretched and elongated inside the cylindrical body; and
wherein the end of the stent body distal from the upper flap and lower flap are facing the side of the plunger.

12. The stent assembly according to claim 10, wherein the application device further comprises:
a thread; and
a cap;
wherein the thread is located at the end of the application device where the stent body exits the application device upon implant of the stent body, and enables placement of the cap in a location that seals the inner part of the cylindrical body.

13. The stent assembly according to claim 10 further comprising the heart valve;
wherein the application device has a diameter large enough to keep the stent body with the heart valve with only the upper and lower flaps compressed within the application device.

14. The stent assembly according to claim 10, wherein the application device has an anatomical shape.

15. The stent assembly according to claim 3, wherein the biological valve comprises one of a bovine and a pig pericardium.

16. The stent assembly according to claim 6, wherein the claws are capable of piercing heart tissue at a location of an implanted stent body.

17. The stent assembly according to claim 8 further comprising an oval or annuloplasty ring placed between the upper flap and lower flap on an outer part of the stent body;
wherein the hooks are capable of grappling muscle at a location of an implanted stent body, facilitating the positioning and fixing of the stent body to the ring.

18. The stent assembly according to claim 9, wherein the metallic segments aid in providing mechanical resistance of the upper and lower flaps.

19. The stent assembly according to claim 11, wherein the application device further comprises:
a thread; and
a cap;
wherein the thread is located at the end of the application device where the stent body exits the application device upon implant of the stent body, and enables placement of the cap in a location that seals the inner part of the cylindrical body.

20. The stent assembly according to claim 11 further comprising the heart valve;
wherein the application device has a diameter large enough to keep the stent body with the heart valve with only the upper and lower flaps compressed within the application device.

21. The stent assembly according to claim 11, wherein the application device has an anatomical shape.

22. The stent assembly according to claim 14, wherein the application device has an ogive shape that one or both facilitates and enables movement of the application device inside veins and arteries.

23. The stent assembly according to claim 21, wherein the application device has an ogive shape that one or both facilitates and enables movement of the application device inside veins and arteries.

24. A stent assembly comprising:
an upper flap;
a lower flap; and
an adapter;
wherein the lower flap is in proximity to and parallel to the upper flap;
wherein both the upper and lower flaps are made of wired material and at least partially coated with an inert material, which allows physiological movement of the mitral annulus and fitting of the adapter; and
wherein the adapter is used to support a heart valve within the stent assembly between both the upper and lower flaps.
